(19) United States
(12) Patent Application Publication
(10) Pub. No.: US 2018/0280665 A1
(43) Pub. Date: Oct. 4, 2018

(54) Over-The-Wire Balloon Catheter and Related Systems and Methods

(71) Applicant: QXMedical, LLC, Roseville, MN (US)

(72) Inventors: Fernando Di Caprio, St. Paul, MN (US); Gianfranco Panarello, Montreal (CA)

(21) Appl. No.: 15/928,918

(22) Filed: Mar. 22, 2018

Related U.S. Application Data
(60) Provisional application No. 62/475,011, filed on Mar. 22, 2017.

(51) Int. Cl. A61M 25/10; A61M 25/09; A61B 5/0215

(52) U.S. Cl. CPC A61M 25/10; A61M 25/09; A61M 2025/1081; A61M 2025/1095; A61M 2025/107; A61B 5/0215

(57) ABSTRACT

Disclosed herein are improved over-the-wire balloon catheters having at least one of a lockable structure that is removably engageable with a guidewire, a partial-flow opening in the catheter body to allow fluid flow through and out of the guidewire lumen, and a partial-flow channel defined in the inflatable body of the catheter, or some combination thereof. Also disclosed herein are methods of using fluid pressure detection devices in combination with the catheter embodiments to detect fluid pressure distal of the inflatable body.

### CROSS-REFERENCE TO RELATED APPLICATION(S)

This application claims the benefit under 35 U.S.C. § 119(e) to U.S. Provisional Application 62/475,011, filed Mar. 22, 2017 and entitled “Over-The-Wire Balloon Catheter,” which is hereby incorporated herein by reference in its entirety.

### FIELD OF THE INVENTION

The various embodiments disclosed or contemplated herein relate to cardiopulmonary intervention technologies, and more specifically to balloon catheters and related systems and methods.

### BACKGROUND OF THE INVENTION

Medical balloon catheters are used for a variety of applications, including occlusion, angioplasty, stent delivery, stent expansion, drug delivery, delivery of therapeutic or diagnostic fluids, thrombus removal, and many other methods and procedures.

One well-known design of a balloon catheter is an “over-the-wire” (or “OTW”) design. An OTW balloon catheter has a guidewire lumen defined along the length of the catheter to accommodate a guidewire such that the catheter can be threaded onto and advanced over the previously-placed guidewire and advanced to its desired location in the vasculature. In addition, such a balloon catheter also has an inflation/deflation lumen that is in fluidic communication with the expandable balloon such that the inflation/deflation lumen can be used to inflate and deflate the balloon.

One known medical procedure typically performed with a balloon catheter is called Resuscitative Endovascular Balloon Occlusion of the Aorta (“REBOA”). A REBOA catheter is designed for use in the emergency and critical care environment and is optimized to support rapid and immediate hemorrhage control. The catheter is designed to be safely and effectively placed with or without the aid of medical imaging if none is available. Hemorrhage due to trauma is the leading preventable cause of death in the military and civilian setting, accounting for up to 90% of potentially preventable deaths. A significant majority of civilian and combat-related mortality caused by traumatic hemorrhage occurs before reaching definitive care. Therefore, early hemorrhage control as a bridge to definitive surgical care may yield a large survival advantage.

REBOA is indicated for traumatic life-threatening hemorrhage below the diaphragm in patients in hemorrhagic shock who are unresponsive or transiently responsive to resuscitation. The balloon catheter may be inflated at the distal aorta for control of severe intra-abdominal or retroperitoneal hemorrhage, severe pelvic, junctional, or proximal lower extremity hemorrhage, or those with traumatic arrest. The inflated balloon catheter occludes blood flow for control of the hemorrhage which simultaneously starves down-stream critical organs of oxygen and nutrients. One method to minimize ischemia to the critical organs is the partial inflation of the balloon allowing a small volume of blood to bypass the balloon by flowing between the balloon and the arterial wall. This technique can result in migration of the balloon catheter which may cause intimal injury if the balloon is not completely deflated or is reinflated in the iliac vessels.

Typically, the procedure must be performed quickly to prevent the patient from bleeding to death. Known devices used for REBOA include a traditional OTW occlusion balloon, a fixed-wire balloon, or a non-wire based balloon catheter. One disadvantage of known OTW catheters is that they may be too time consuming to be effective in the REBOA procedure, because use of known OTW catheters requires an additional step: placement of a guidewire into the aorta prior to the introduction of the known balloon catheter. One disadvantage of the fixed-wire catheter and the non-wire based catheter is that they generally lack sufficient “trackability” to navigate potential calcification and tortuosity as the catheter is advanced through a vessel, where “trackability” is any combination of stiffness and flexibility characteristics in the catheter that are sufficient to provide for navigation of such calcification and tortuosity.

There is a need in the art for an improved balloon catheter and related systems and methods.

### BRIEF SUMMARY OF THE INVENTION

Discussed herein are various over-the-wire balloon catheter embodiments with various features or components, including a lockable structure that is removably engageable with a guidewire, a partial-flow opening in the catheter body to allow fluid flow through and out of the guidewire lumen, and/or a partial-flow channel defined in the inflatable body of the catheter.

In Example 1, a balloon catheter comprises a catheter body, a guidewire lumen defined through a length of the catheter body, an inflatable body disposed on the catheter body, and a lockable structure disposed adjacent to a proximal end of the catheter body, wherein the lockable structure is constructed and arranged to be removably engageable with a guidewire disposed within the guidewire lumen.

Example 2 relates to the balloon catheter according to Example 1, wherein the lockable structure comprises a lockable clamp or a lockable collar.

Example 3 relates to the balloon catheter according to Example 1, wherein the lockable structure comprises a slidable lockable structure or a rotatable lockable structure.

In Example 4, a balloon catheter comprises a catheter body, a guidewire lumen defined through a length of the catheter body, an inflatable body disposed on the catheter body, and at least one opening defined in the catheter body, wherein the at least one opening is in fluid communication with the guidewire lumen and is proximal to the inflatable body and distal to a proximal end of the catheter body.

Example 5 relates to the balloon catheter according to Example 4, wherein the at least one opening is a slit.

Example 6 relates to the balloon catheter according to Example 4, wherein the at least one opening comprises at least two openings.

In Example 7, a balloon catheter comprises a catheter body, a guidewire lumen defined through a length of the catheter body, an inflatable body disposed on the catheter body, a partial-flow channel defined in an inflated configuration of the inflatable body, and an elongate tension member operably coupled with the inflatable body, wherein the elongate tension member is movable between a fully tensioned position and a fully non-tensioned position.

Example 8 relates to the balloon catheter according to Example 7, wherein the partial-flow channel has a longitudinal axis parallel to a longitudinal axis of the catheter body.

Example 9 relates to the balloon catheter according to Example 7, wherein the partial-flow channel is defined by an outer surface of the inflatable body.

Example 10 relates to the balloon catheter according to Example 9, wherein the partial-flow channel is further defined by an inner surface of a blood vessel, wherein the inflatable body is disposed within the blood vessel.

Example 11 relates to the balloon catheter according to Example 7, further comprising an elongate structure disposed along a length of the inflatable body.

Example 12 relates to the balloon catheter according to Example 11, wherein the partial-flow channel is formed in part by the elongate structure.

Example 13 relates to the balloon catheter according to Example 11, wherein the elongate structure is attached at a first end to the catheter body and is attached at a second end to the catheter body.

Example 14 relates to the balloon catheter according to Example 11, wherein a tension of the elongate structure can be adjusted, whereby a size of the partial-flow channel can be adjusted.

Example 15 relates to the balloon catheter according to Example 7, wherein the elongate tension member is disposed within the partial-flow channel.

Example 16 relates to the balloon catheter according to Example 15, wherein movement of the elongate tension member toward the fully tensioned position causes the partial-flow channel to increase in size.

Example 17 relates to the balloon catheter according to Example 15, wherein movement of the elongate tension member toward the fully non-tensioned position causes the partial-flow channel to decrease in size.

Example 18 relates to the balloon catheter according to Example 7, wherein the elongate tension member is lockable at any position from the fully tensioned position to the fully non-tensioned position

In Example 19, a method of performing a cardiovascular intervention procedure comprises positioning an introducer sheath into a blood vessel, positioning a balloon catheter into the introducer sheath, wherein a guidewire is disposed within a guidewire lumen of the catheter body in a retracted position, advancing the guidewire distally out of a distal end of the catheter body, locking the guidewire in relation to the catheter body via a lockable structure, advancing the balloon catheter and guidewire distally to a desired location, and inflating an inflatable body disposed on the catheter body.

Example 20 relates to the method according to Example 19, further comprising unlocking the guidewire in relation to the catheter body upon encountering an obstruction, advancing the guidewire in relation to the catheter body past the obstruction, and advancing the balloon catheter distally after advancing the guidewire past the obstruction.

Example 21 relates to the method according to Example 19, further comprising measuring a blood pressure distal of the inflatable body with a pressure measuring device.

Example 22 relates to the method according to Example 21, further comprising removing the guidewire from the guidewire lumen, and positioning the pressure measuring device in communication with the guidewire lumen.

Example 23 relates to the method according to Example 21, further comprising positioning the pressure measuring device in communication with a second lumen defined in the catheter body.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a side view of an over-the-wire balloon catheter, according to one embodiment.

FIG. 1B is an expanded view of the proximal end of the over-the-wire balloon catheter of FIG. 1A, according to one embodiment.

FIG. 1C is a side view of the over-the-wire balloon catheter of FIG. 1A depicting the guidewire in the retracted position, according to one embodiment.

FIG. 1D is a cross-sectional view of the catheter body of the catheter of FIG. 1A, according to one embodiment.

FIG. 2A is a side view of another over-the-wire balloon catheter, according to a further embodiment.

FIG. 2B is an expanded cross-sectional view of a portion of the over-the-wire balloon catheter of FIG. 2A, according to a further embodiment.

FIG. 2C is an expanded side view of a proximal portion of the over-the-wire balloon catheter of FIG. 2A, according to a further embodiment.

FIG. 3A is a side view of a proximal end of an expanded balloon on a balloon catheter, according to one embodiment.

FIG. 3B is a side view of a distal end of the expanded balloon of FIG. 3A, according to one embodiment.

FIG. 3C is an expanded side view of a proximal portion of the over-the-wire balloon catheter of FIG. 3A, according to a further embodiment.

### DETAILED DESCRIPTION

The various embodiments disclosed or contemplated herein relate to an OTW balloon catheter that has a guidewire placed (or incorporated) in a guidewire lumen of the balloon catheter. In certain implementations, the guidewire is held in place by a lockable clamp on the proximal end of the balloon catheter. According to other implementations, the balloon catheter includes a feature that allows for partial fluid flow past the inflated balloon during use. Of course, certain catheter examples will have both the lockable clamp and a partial flow feature. In accordance with certain implementations, the various catheter embodiments disclosed or contemplated herein can be used for REBOA procedures.

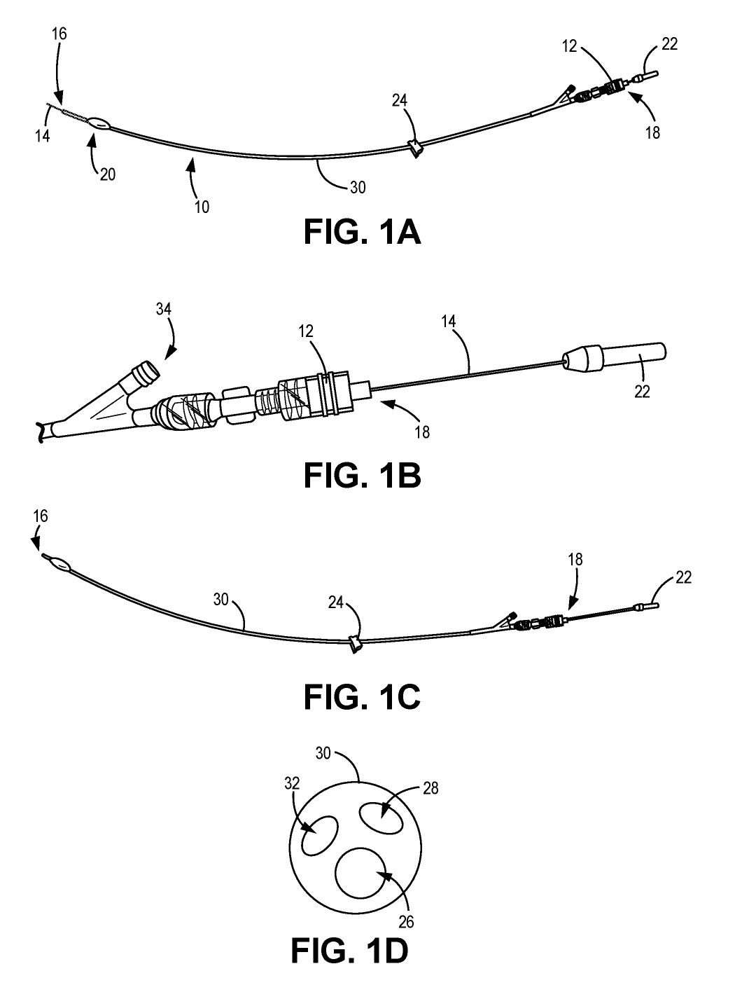

One embodiment of an OTW balloon catheter 10 is shown in FIGS. 1A-1D. The catheter 10 has a lockable clamp 12, a guidewire 14 disposed through a guidewire lumen 26 (as best shown in FIG. 1D) defined through the length of the catheter body 30, a distal opening 16 in the body 30 providing fluid access to the guidewire lumen 26, a proximal opening 18 to the guidewire lumen 26, an expandable balloon 20, an inflation/deflation lumen 28 (as best shown in FIG. 1D) defined along the length of the catheter 10 from the proximal inflation port 34 (as best shown in FIG. 1B) to the balloon 20 and is in fluid communication with the balloon 20, and a third lumen 32 (as best shown in FIG. 1D) defined along some portion of the length of the catheter 10 depending on the specific function of the lumen 32 in the specific embodiment. In one exemplary embodiment, the third lumen 32 is a blood pressure monitoring lumen 32 that can be used in conjunction with a monitoring device as described in additional detail below. Alternatively, the third lumen 32 can be an elongate tension structure lumen 32 that can be used in conjunction with a tension structure coupled to the balloon as described in further detail in relation to other embodiments discussed below. In a further alternative, the third lumen 32 can be used for any known purpose and in conjunction with any known component or device that is typically used in conjunction with a balloon catheter such as catheter 10. The inflation/deflation lumen 28 is used to inflate and deflate the balloon 20 and the guidewire lumen 26 is used to house the guidewire 14 during catheter insertion into the patient's body and facilitate guidewire manipulation during tracking.

In certain implementations, the guidewire 14 can have a handle 22 at or near the proximal end of the guidewire 14, which can aid in handling or torqueing the guidewire 14. Further, it is understood that the guidewire 14 can have various distal tip configurations. In FIGS. 1A-1C, the distal tip is straight, but it can also have various pre-set shapes or configurations as is known in the art such as a “j-tip” configuration. Alternatively, the distal tip can also be shaped during the procedure to have the ideal curvature for the specific vasculature. The shaped guidewire tip combined with the capability to torque or twist the guidewire provides optimal control of the guidewire to selectively steer to a vessel location.

An expanded view of the proximal end of the catheter 10 and the lockable clamp 12 is depicted in FIG. 1B. In this implementation, the clamp 12 is positioned at or near the proximal opening 18 of the catheter 10. The clamp 12 may be moved between a locked configuration in which the clamp 12 fixes the guidewire 14 in place in relation to the catheter 10 and an unlocked configuration in which the guidewire 14 is released such that the guidewire 14 can be moved (manipulated, torqued, repositioned, removed, etc.) in relation to the lumen (not shown) of the catheter 10 and thus along the vasculature as needed.

In one embodiment as shown, the clamp 12 has a slidable locking function in which a user can urge the clamp 12 (distally/proximally) into the locked configuration to fix the guidewire 14 in place and can urge the clamp 12 (distally/proximally) into the unlocked configuration to release the guidewire 14. Alternatively, the clamp 12 can have a rotating locking function, or any other known mechanism for fixing the guidewire 14 in relation to the catheter 10. In certain embodiments, the clamp 12 can be a known Touhy-Borst or hemostasis valve connector 12.

When the clamp 12 is in the locked configuration, the clamp 12 in certain embodiments can be swiveled or twisted in relation to the catheter 10, thereby making it possible for the user to torque or twist the guidewire 14 in relation to the catheter 10 while restraining the guidewire 14 from moving longitudinally in relation to the catheter 10. It is understood that the torqueing or twisting of the guidewire 14 provides control of the wire 14 to selectively steer the catheter 10/wire 14 combination to a specific vessel location.

In certain embodiments, the catheter 10 can also have a shaft marker 24, as best shown in FIGS. 1A and 10. The marker 24 can be a radiopaque marker 24 that allows the user to insert and advance the balloon catheter 10 to the desired vascular position with or without medical imaging (including, for example, fluoroscopy). In this specific example, the marker 24 is slidable/positionable along the shaft of the catheter body 30 and can be positioned to indicate an insertion or depth landmark. It is understood that any catheter embodiment disclosed or contemplated herein can have a shaft marker 24.

The use of the clamp 12 to fix the guidewire 14 to the catheter 10 allows for advancement of the balloon catheter 10 and guidewire 14 as a single assembly. When initially inserting the catheter 10 through an introducer sheath with a hemostasis valve, it may be helpful or necessary to retract the distal end of the guidewire 14 proximally into the lumen (not shown) of the catheter 10. FIGS. 1B and 10 depict the guidewire 14 in the retracted position. Having the guidewire 14 exposed out of the distal end of the catheter 10 when initially introducing the catheter 10/guidewire 14 assembly may cause the guidewire 14 to kink or break. Once the distal tip of the catheter 10 is advanced into or through the introducer sheath, the guidewire 14 may be urged distally in relation to the catheter 10 to a position in which the distal end of the guidewire 14 extends distally out of the distal end of the catheter 10, as shown in FIG. 1A.

In another implementation, the guidewire lumen 26 of the catheter 10 can be used to provide access for the performance of any other known procedure requiring a lumen, such as, for example, monitoring blood pressure. That is, a user can unlock the clamp 12 and retract the guidewire 14 proximally from the catheter 10 until the guidewire 14 is fully removed from the lumen 26. Once the guidewire 14 is removed, in the specific blood pressure monitoring example, a pressure monitor (not shown) can be coupled to the proximal end of the catheter 10 such that the monitor is in communication with the lumen 26, thereby allowing for measurement of blood pressure in the vessel in which the distal end of the catheter 10 is positioned. Alternatively, any other known device or procedure that requires or uses a lumen of a balloon catheter can be utilized with any of the catheter embodiments herein.

According to another embodiment, certain catheter embodiments disclosed or contemplated herein can have a partial flow feature that allows fluid to bypass the inflated balloon of the catheter that is positioned in the vessel.

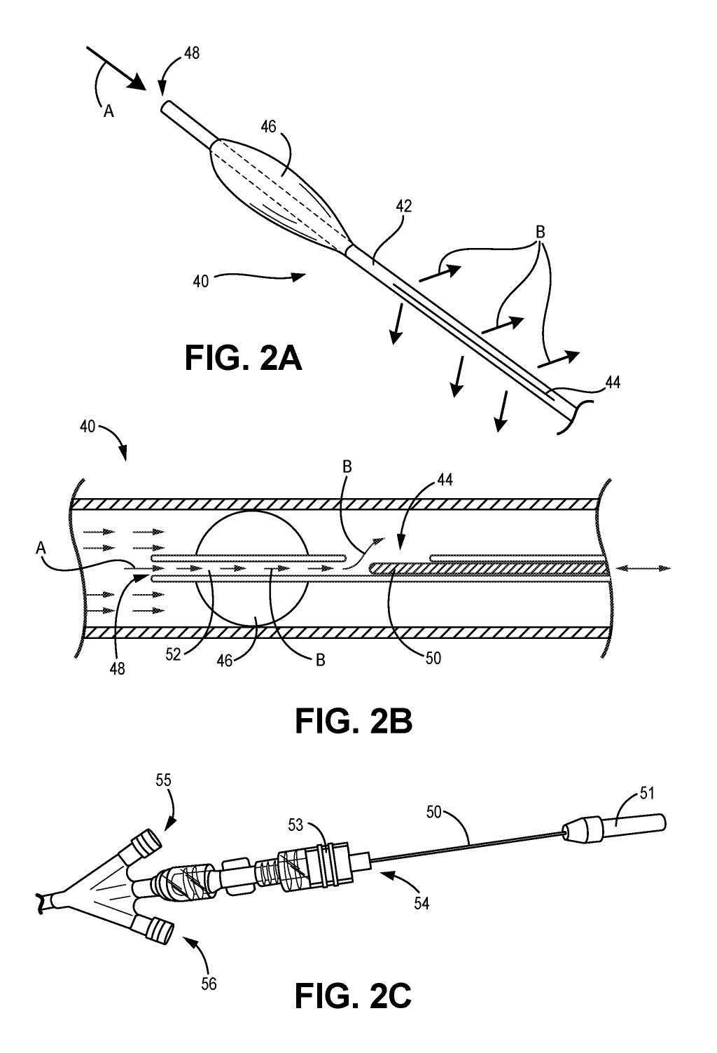

FIGS. 2A-2C depict another OTW balloon catheter 40 embodiment having a partial flow feature, according to one embodiment. More specifically, in this specific implementation as best shown in FIG. 2A, the partial flow feature is a slit (or “slot”) 44 defined in the shaft (or “body”) 42 of the catheter 40 along a length of the shaft 42 that is proximal to the balloon 46. The slit 44 provides fluidic access to the guidewire lumen 52 (as best shown in FIG. 2B) defined in the catheter 40 and thus to the distal opening 48 of the catheter 40. As such, fluid that flows in the proximal direction into the distal opening 48 of the catheter 40 as represented by the arrow A and through the guidewire lumen 52 can exit the guidewire lumen 52 at the slit 44 as represented by arrows B. Alternatively, the slit 44 can allow for flow in the other direction as well. That is, when the catheter 40 is inserted in the opposite orientation to the flow such that the fluid flows from the proximal end of the catheter 40 toward the distal end, the fluid flows through the slit 44 and into the guidewire lumen 52 of catheter 40, through the guidewire lumen 52 and exits the lumen at the distal opening 48. Alternatively, the catheter 40 can have two or more slits. In a further alternative, the catheter 40 can have any type of opening and any number of such openings formed in the shaft 42 proximal to the balloon 46 but distal to the proximal end of the catheter 40 such that the opening will allow fluid to pass from the guidewire lumen 52 out of the opening.

It is understood that the catheter 40 (and catheter 60 below) can have the same or similar components, features, and functionality as those described above with respect to catheter 10, except as otherwise set forth herein.

As best shown in FIG. 2C, the catheter 40 in certain implementations can also have a lockable clamp 53, a proximal opening (or “port”) 54 to the guidewire lumen 52, an inflation/deflation port 55 in fluid communication with an inflation/deflation lumen (not shown), and a pressure monitoring port 56 in fluid communication with a pressure monitoring lumen (not shown). In addition, the guidewire 50 can have a handle 51. Alternatively, the catheter 40 can have any combination of these ports depending on the various alternative features incorporated into the catheter 40 as discussed in the various embodiments herein.

According to one embodiment as shown in FIG. 2B, a guidewire 50 disposed in the guidewire lumen 52 of the catheter 40 can help to control the partial flow of fluid through the guidewire lumen 52 and out of the slit 44. More specifically, if the guidewire 50 is positioned in the guidewire lumen 52 such that the guidewire 50 extends from the distal opening 48 of the catheter 40, the guidewire 50 can be retracted (urged proximally) along the guidewire lumen 52 until the distal end of the guidewire 50 is disposed proximal of the slit 44 as shown in FIG. 2B such that a greater amount of fluid can flow proximally along the guidewire lumen 52 and out of the slit 44 (as represented by arrows A). The guidewire 50 may further have one or more ring-like structure(s) (not shown) or other similar physical features along the length of the guidewire 50 that restrict flow within the guidewire lumen 52 and thereby provide even greater control of by-pass flow. According to various embodiments, the ring-like structure(s) can consist of elevated circumferential steps, circumferential O-rings, or other designs that minimize the gap between the guidewire 50 and the guidewire lumen 52 wall to minimize fluid bypass.

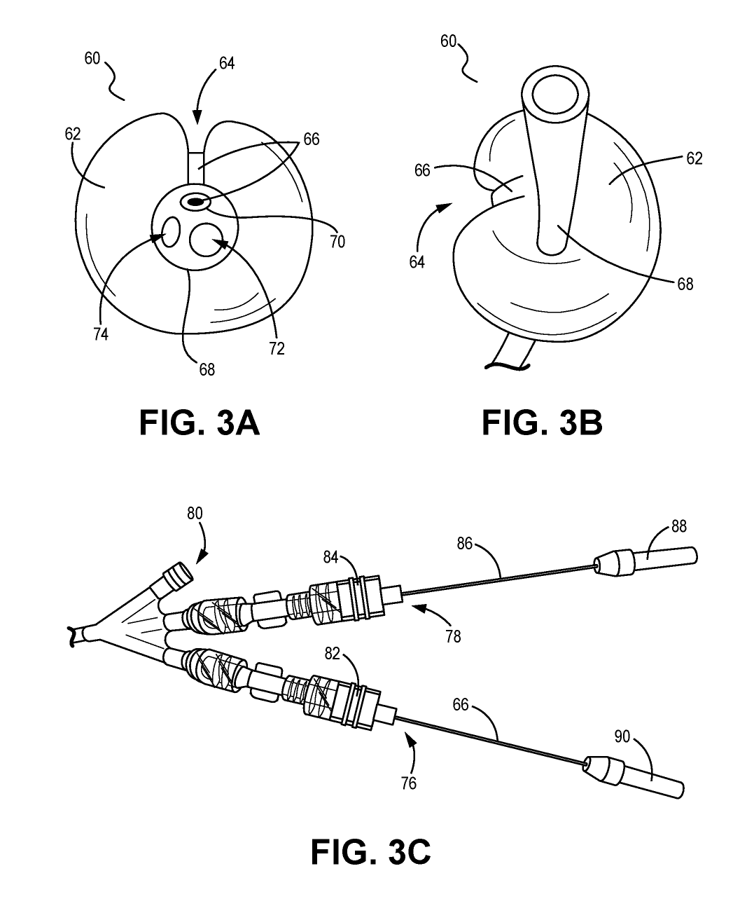

Another implementation of a catheter 60 with a partial flow feature is depicted in FIGS. 3A-3C. More specifically, the catheter 60 in these figures has a balloon 62 that has a channel (also referred to herein as a “partial-flow channel,” “bypass channel,” “partial-flow gap,” or “bypass gap”) 64 or other similar feature defined around the circumference and extending down the longitudinal axis of the balloon 62 when it is inflated. In other words, the balloon 62 is designed or constrained to inflate such that the outer surface of the balloon 62 does not achieve a full 360 degrees of circumferential contact with the inner wall of the vessel in which it is positioned. Put yet another way, the inflated balloon 62 in this embodiment defines a small lumen (also referred to herein as a “partial-flow lumen” or “bypass lumen”) 64 between the balloon 62 and the inner surface or wall of the blood vessel in which the catheter 60 is positioned during use.

In this specific exemplary embodiment as shown, the balloon 62 has an elongate member 66 that is attached at one end to the shaft 68 on one side of the balloon 62 and extends from that side along the balloon 62 to the other side of the balloon 62 where it is attached to the shaft 68. Alternatively, proximal of the balloon 62, the elongate member 66 passes through an opening in the catheter body 68 and into a tension member lumen 70 and extends proximally out of the proximal end of the catheter 60 as discussed in further detail below. In one embodiment, the elongate member 66 is a wire, ribbon, or cord. Alternatively, the elongate member 66 can be any elongate member that can be used to restrain the balloon 62 from expanding fully as shown. According to certain alternative embodiments, the tension of the elongate member 66 can be independently controlled by a user to adjust the size of the channel 64 (and thus the amount of resulting bypass flow). For example, in the instant embodiment, the elongate tension member 66 is adjustable by urging the proximal end of the member 66 proximally or distally as discussed in further detail below.

Alternatively, a constraining material or elongate member can be incorporated into the structure of the balloon 62. In a further alternative, two or more elongate members according to any embodiment herein can be used. In yet another alternative, any other structure or balloon design—other than an elongate member—that results in a partial-flow channel or partial-flow lumen is contemplated herein, such as adhering or otherwise attaching the outer circumference of balloon 62 to the shaft 68 at a circumferential point extending down a longitudinal axis parallel to a longitudinal axis of the catheter body 68.

As best shown in FIG. 3A, the catheter 60 in certain implementations can have three lumens 70, 72, 74 defined through certain lengths of the catheter body 68. For example, in this specific embodiment, the catheter 60 has an tension member lumen 70, a guidewire lumen 72, and an inflation/deflation lumen 74. In this implementation, as mentioned above, the tension member lumen 70 is configured to receive or have positioned therethrough the elongate tension member 66 such that the tension member 66 extends from the balloon 62 as described above proximally through the lumen 70 and out of the proximal end of the catheter 60 such that a user can urge the tension member 66 proximally or distally to control the tension of the member 66 as discussed above. In certain embodiments, the tension member 66 has a handle 90. The guidewire lumen 72 receives the guidewire 86, which can also have a handle 88.

As best shown in FIG. 3C, the catheter 60 according to some embodiments has a proximal end with three ports 76, 78, 80. More specifically, the catheter 60 can have a tension member port 76, a guidewire port or opening 78, and an inflation/deflation port 80. The guidewire port 78 is in fluid communication with the guidewire lumen 72, the tension member port 76 is in fluid communication with the tension member lumen 70, and the inflation/deflation port 80 is in fluid communication with the inflation/deflation lumen 74. In this embodiment, both the tension structure port 76 and the guidewire port 78 have lockable clamps 82, 84 similar to those discussed above that operate in substantially the same fashion. Alternatively, the catheter 60 can have any combination of these ports depending on the various alternative features incorporated into the catheter 60 as discussed in the various embodiments herein.

In use, a user can grasp the tension member 66 (or the handle 90 thereof) and urge the tension member 66 proximally or distally to adjust the size of the channel 64 in the balloon 62. That is, if the tension member 66 is urged proximally to its fully tensioned position, the tension member 66 is drawn taut where it is coupled to the balloon 62 and thereby causes the channel 64 to increase to its largest size, thereby allowing the greatest amount of fluid to pass. If the tension member 66 is released or is urged distally to its fully non-tensioned position, the tension member 66 loses all tension where it is coupled to the balloon and thereby causes the channel 64 to decrease to its smallest size or to be completely eliminated such that no channel 64 exists. Further, the user can position the tension member 66 at any point between the fully tensioned position and the fully non-tensioned position to adjust the size of the channel 64 to any size between the largest size and the smallest size. In one embodiment, the user can use the lockable clamp 82 to fix the tension member 66, and thus the channel 64, at the desired position.

It is understood that the various embodiments contemplated herein include some implementations relating to catheters having both a lockable guidewire clamp and a partial flow feature. As such, any of the components of the various embodiments disclosed or contemplated herein can be combined with any other components disclosed or contemplated herein in any fashion or combination.

In use in a REBOA procedure, the various catheter embodiments herein do not require the placement of a guidewire prior to introduction of the catheter. Further, the various embodiments have “trackability” to navigate all types of vessels and obstructions that may be found therein.

Typically, the first step in a REBOA procedure is the placement of an introducer sheath (typically in the patient's femoral artery, brachial artery, or radial artery) to access the aorta using known surgical techniques. Next, the balloon catheter according to any embodiment herein is introduced through the sheath by retracting the guidewire (or positioning the guidewire in a retracted position) into the distal end of the balloon catheter and pushing the balloon catheter tip distally through the sheath. At that point, the guidewire is advanced distally in relation to the catheter to extend out the distal end of the balloon catheter and locked into place using the lockable clamp. The entire assembly is then advanced to its desired location. In the event that the advancement of the catheter is blocked by an obstruction or other vessel feature such as calcification or a branch artery, the catheter is retracted (urged proximally) slightly, the guidewire is unlocked using the clamp, and then the guidewire is urged distally in relation to the catheter. Once the guidewire gets across the obstruction, the balloon catheter can then be urged distally. Once in place, the balloon is inflated, the blood flow is occluded, and the desired procedure is performed. Alternatively in the event that the advancement of the catheter is obstructed, the guidewire can be torqued or twisted to selectively steer around or through the obstruction with the guidewire locked in the rotating clamp.

Other, alternative steps can also be performed as mentioned above. For example, in certain implementations, the blood pressure can be monitored by removing the guidewire and coupling a monitor to the proximal end of the catheter.

In a further alternative in which the catheter has the opening(s) proximal to the balloon as shown, for example, in FIGS. 2A and 2B, the guidewire can be retracted to allow bypass flow proximally past the occluded balloon area. In yet another alternative in which the catheter has a partial-flow channel in the circumference of the balloon as shown, for example, in FIGS. 3A and 3B, bypass past the occluded balloon area can be induced as discussed above.

Although the present invention has been described with reference to preferred embodiments, persons skilled in the art will recognize that changes may be made in form and detail without departing from the spirit and scope of the invention.

What is claimed is:

1. A balloon catheter comprising:
(a) a catheter body;
(b) a guidewire lumen defined through a length of the catheter body;
(c) an inflatable body disposed on the catheter body; and
(d) a lockable structure disposed adjacent to a proximal end of the catheter body, wherein the lockable structure is constructed and arranged to be removably engageable with a guidewire disposed within the guidewire lumen.

2. The balloon catheter of claim 1, wherein the lockable structure comprises a lockable clamp or a lockable collar.

3. The balloon catheter of claim 1, wherein the lockable structure comprises a slidable lockable structure or a rotatable lockable structure.

4. A balloon catheter comprising:
(a) a catheter body;
(b) a guidewire lumen defined through a length of the catheter body;
(c) an inflatable body disposed on the catheter body; and
(d) at least one opening defined in the catheter body, wherein the at least one opening is in fluid communication with the guidewire lumen and is proximal to the inflatable body and distal to a proximal end of the catheter body.

5. The balloon catheter of claim 4, wherein the at least one opening is a slit.

6. The balloon catheter of claim 4, wherein the at least one opening comprises at least two openings.

7. A balloon catheter comprising:
(a) a catheter body;
(b) a guidewire lumen defined through a length of the catheter body;
(c) an inflatable body disposed on the catheter body;
(d) a partial-flow channel defined in an inflated configuration of the inflatable body; and
(e) an elongate tension member operably coupled with the inflatable body, wherein the elongate tension member is movable between a fully tensioned position and a fully non-tensioned position.

8. The balloon catheter of claim 7, wherein the partial-flow channel has a longitudinal axis parallel to a longitudinal axis of the catheter body.

9. The balloon catheter of claim 7, wherein the partial-flow channel is defined by an outer surface of the inflatable body.

10. The balloon catheter of claim 9, wherein the partial-flow channel is further defined by an inner surface of a blood vessel, wherein the inflatable body is disposed within the blood vessel.

11. The balloon catheter of claim 7, further comprising an elongate structure disposed along a length of the inflatable body.

12. The balloon catheter of claim 11, wherein the partial-flow channel is formed in part by the elongate structure.

13. The balloon catheter of claim 11, wherein the elongate structure is attached at a first end to the catheter body and is attached at a second end to the catheter body.

14. The balloon catheter of claim 11, wherein a tension of the elongate structure can be adjusted, whereby a size of the partial-flow channel can be adjusted.

15. The balloon catheter of claim 7, wherein the elongate tension member is disposed within the partial-flow channel.

16. The balloon catheter of claim 15, wherein movement of the elongate tension member toward the fully tensioned position causes the partial-flow channel to increase in size.

17. The balloon catheter of claim 15, wherein movement of the elongate tension member toward the fully non-tensioned position causes the partial-flow channel to decrease in size.

18. The balloon catheter of claim 7, wherein the elongate tension member is lockable at any position from the fully tensioned position to the fully non-tensioned position

19. A method of performing a cardiovascular intervention procedure, the method comprising:
positioning an introducer sheath into a blood vessel;
positioning a balloon catheter into the introducer sheath, wherein a guidewire is disposed within a guidewire lumen of the catheter body in a retracted position;
advancing the guidewire distally out of a distal end of the catheter body;
locking the guidewire in relation to the catheter body via a lockable structure;
advancing the balloon catheter and guidewire distally to a desired location; and
inflating an inflatable body disposed on the catheter body.

20. The method of claim 19, further comprising:
unlocking the guidewire in relation to the catheter body upon encountering an obstruction;
advancing the guidewire in relation to the catheter body past the obstruction; and
advancing the balloon catheter distally after advancing the guidewire past the obstruction.

21. The method of claim 19, further comprising measuring a blood pressure distal of the inflatable body with a pressure measuring device.

22. The method of claim 21, further comprising:
removing the guidewire from the guidewire lumen; and
positioning the pressure measuring device in communication with the guidewire lumen.

23. The method of claim 21, further comprising positioning the pressure measuring device in communication with a second lumen defined in the catheter body.
